(19) United States
(12) Patent Application Publication
(10) Pub. No.: US 2007/0149657 A1
(43) Pub. Date: Jun. 28, 2007

(54) CORRUGATING ADHESIVES FOR BONDING COATED PAPERS AND METHODS FOR BONDING COATED PAPERS

(76) Inventors: Roman Skuratowicz, Hickory Hills, IL (US); James Linder, Anderson, IN (US); Bryan Turner, Independence, KY (US)

Correspondence Address: NORRIS MCLAUGHLIN & MARCUS, P.A., P O BOX 1018, SOMERVILLE, NJ, 08876, US

(21) Appl. No.: 11/564,349

(22) Filed: Nov. 29, 2006

Related U.S. Application Data
(63) Continuation-in-part of application No. 11/427,141, filed on Jun. 28, 2006.
Application No. 11/427,141 is a continuation of application No. 10/302,111, filed on Nov. 22, 2002, now abandoned.

(51) Int. Cl. D21H 19/54; C08L 5/00; D06P 1/44; C09D 101/18; C08L 1/00; D21H 19/60; A61L 17/12; A61K 8/73; C08K 5/55

(52) U.S. Cl. 524/47; 524/27; 524/28; 524/31; 524/35; 524/45; 524/46; 524/54; 524/55; 524/183; 524/184

(57) ABSTRACT

A corrugating adhesive that improves the bonding of coated papers, including latex coated medium and linerboards. The corrugating adhesive may comprise a hydrocolloid portion of the adhesive that improves tack and bonding and buffers and absorbs caustic in the adhesive allowing higher caustic in the adhesive formula without premature gelling of the secondary starch portion. Also, methods for making waterproof or water resistant corrugated board from corrugated media and paper liner.

### CROSS-REFERENCE TO RELATED APPLICATIONS

This application is a continuation-in-part of U.S. patent application Ser. No. 11/427,141 filed on Jun. 28, 2006, currently pending, which is a continuation of U.S. patent application Ser. No. 10/302,111, filed Nov. 22, 2002, now abandoned. U.S. patent application Ser. No.11/427,141 and U.S. patent application Ser. No. 10/302,111 are both incorporated herein by reference in their entirety.

### BACKGROUND OF THE INVENTION

1. Field of the Invention

The invention pertains to an adhesive composition comprising hydrocolloids, such as hemicellulose, and starch that improves the bonding and waterproofing bonding of coated papers, including papers coated with complex latex coated medium and linerboards. The hydrocolloid portion in the carrier of the adhesive improves tack and bonding and buffers and absorbs caustic in the adhesive allowing higher caustic in the adhesive formula without premature gelling of the secondary starch portion. The caustic concentration in the composition is a function of a critical constant which can be increased with the inclusion of hemicellulose in the composition thereby allowing for more caustic in the adhesive composition.

2. The Prior Art

In the corrugating process, adhesive is commonly applied to the tips of the flutes of a corrugated medium. Then a non-corrugated flat paper liner is applied against the adhesive coated flutes as they pass between a corrugating roll and a pressure roll. The resulting product has the corrugating medium on one side and a flat liner on the other side and is called a single-face portion. The single-face portion may be used “as is” (called a “single-face” board) or adhesive may be applied to the flute tips of the single-face portion and a second flat sheet can be applied in the same manner as the first in what is called a “double-face” or a “double-back” operation. The second liner sheet is treated with heat and reduced pressure (relative to the pressure used to make a single-face portion) immediately following contact with the adhesive coated flutes as they pass between a corrugating roll and a pressure roll.

Starch-based adhesives which can be of the carrier, no-carrier and carrier no-carrier type are commonly used in processes for manufacturing corrugated paper board. In carrier type adhesives, a portion of the starch (or dextrin) forms a carrier, often known as the gelatinized phase, which suspends the balance of the starch which is in an ungelatinized state. Under conditions of heat and pressure, the ungelatinized starch is rapidly hydrated and gelatinized to increase quickly the viscosity and adhesivity of the adhesive composition. In no-carrier type adhesives, all of the starch is slightly cooked or swollen with heat and caustic soda for viscosity. Finally, carrier no-carrier type adhesives have a portion of the starch which forms a carrier and is responsible for about one half of the viscosity and the remaining viscosity is obtained by slightly swelling the uncooked starch.

Starch based corrugating adhesives of the carrier, no-carrier and carrier-no-carrier type comprising added hemicellulose are described U.S. Pat. No. 5,358,559. Adhesive compositions where the hemicellulose is extracted from corn fiber in-situ during the process of preparing the carrier phase are discussed in U.S. Pat. No. 6,368,443 B1.

Waterproof and chemically resistant corrugated boxes are, traditionally, wax coated boxes that are not recyclable and add disposal cost to the end user. More recently, specially coated papers, like complex latex coated medium and linerboards, such as those described in U.S. Pat. Nos. 5,393,566; 5,429,294 and 5,858,173, have been developed that result in recyclable waterproof and chemically resistant boxes that can replace wax coated boxes. Bonding water proof and chemically resistant media with traditional adhesives encounters drawbacks. Traditional corrugating adhesives rely on waterproofing resins, such as ketone aldehyde or phenolic resins, to provide water resistance to these adhesives. These waterproof corrugating adhesives have been found to fail in waterproofing many new water resistant coated papers, primarily when the adhesive bonds merely to the coating surface and not to the paper fibers itself.

The hydrocolloid portion of the adhesive provides improved tack and bonding particularly in the carrier of the adhesive. Also, when the hydrocolloid is a fiber source rich in hemicellulose, both the hemicellulose and the cellulose portion buffer and absorb caustic in the adhesive, which allows the adhesive to have higher caustic in the formula without premature gelling of the secondary starch. Other hydrocolloids will behave similar to cellulose and hemicellulose to various degrees. The higher caustic improves bonding by reacting with the coating surface at the bond point and enabling penetration of the adhesive and waterproofing resin into the paper surface.

In the present Specification all parts and percentages are on a weight by weight basis based on the total amount of components in a composition of matter, unless otherwise specified. Thus, compositional amounts set forth herein by weight without any further qualification or by weight of the composition are on a weight by weight basis based on the total amount of components in the composition.

### SUMMARY OF THE INVENTION

The corrugating adhesive comprises starch, hydrocolloid, caustic, boron containing compound, waterproofing or water resistant resins and water. Optionally, the corrugating adhesive comprises other additives and fillers, such as biocides, defoamers, and performance enhancing additives, including liquid additives, polyvinyl alcohol or latexes and the like.

The hydrocolloid is preferably hemicellulose, which has a cellulose fraction, however, any hydrocolloid with similar chemistry to hemicellulose can be used, such as those selected from the group consisting of gum arabic, xanthan gum, gum karaya, tragacanth, sodium alginates, carageenan, Guar gum, Locus bean gum, tara, pectins, gellan, cellulose derivatives such as carboxymethyl, methyl or ethyl cellulose, microcrystalline cellulose, or other polysaccharide type hydrocolloids. Separated hemicellulose or cellulose fractions can also be used.

The corrugating adhesive can be made by use of a primary and secondary mixer, single tank mixer, high shear mixer, Minocar system or other typical preparation system. Hydrocolloid can be added to the primary or secondary mixer in the preparation of a carrier type corrugating adhesive or it can be added at any state in the preparation of a carrier no-carrier type adhesive. It also may be added to a prepared adhesive in dry or liquid form prior to application to the corrugator. In the preferred embodiment, wherein the hydrocolloid is obtained from corn fiber, hemicellulose may be extracted in-situ from corn fiber with cellulose also present.

The invention also pertains to a method for bonding water proof or chemically resistant papers, such as bonding coated corrugated media and liners. The adhesive is suited for bonding latex coated medium and linerboards, although the adhesive can be used for other types of coated media, such as papers coated with wax, other traditional coating materials, more recent coating materials and the like. The method can be used to make single-face or double-face coated products. The process is an improvement over conventional corrugating processes in a number of ways. For instance, the hydrocolloid portion (e.g. hemicellulose) provides improved tack and bonding in the carrier portion of the adhesive. Also, in the case of corn fiber as a hydrocolloid source, both the hemicellulose and the cellulose portion buffer and absorb caustic in the adhesive. This allows the adhesive to have higher caustic in the formula without gelling the secondary starch portion prematurely. The higher caustic improves bonding by reacting with the coating surface at the bond point, thereby enabling penetration of the adhesive and waterproofing or water resistant resin into the paper surface and firmly bonding the coated corrugating medium and liner together, including latex coated medium and linerboards, without any loss in the resistant properties of the coated papers.

As discussed above, coated papers, such as water proof or chemically resistant papers, specifically those with acrylic or other synthetic coatings to improve water or chemical resistance, are very difficult to bond with starch based corrugating adhesives and it is even more difficult to make a waterproof bond because the coating interferes with adhesive adherence and penetration. The coating on the papers also interferes with the action of waterproofing resins used in adhesives to obtain water resistant bonds. Coatings to improve water or chemical resistance, such as acrylic or other synthetic coatings, can be weakened and penetrated by incorporating higher than normal amounts of alkali in adhesives and this weakening improves adherence and allows the adhesive to penetrate past the coatings and bond better with paper fibers themselves. In typical corrugating adhesive, however, this amount of high alkali is not possible because it will swell and gel uncooked secondary starch, causing the adhesive to set up to a solid material.

### DETAILED DESCRIPTION OF THE INVENTION

The corrugating adhesives are an aqueous emulsion comprising starch, hydrocolloid, caustic, borax, waterproofing or water resistant resins and water. The corrugating adhesive may comprise from about 15% to about 45% by weight starch, up to about 40%, including up to about 20%, by weight hydrocolloid, caustic in an amount of about 0.75% to about 2.25% by weight, up to about 2% by weight boron containing compound, from about 0.5% to about 5% by weight waterproofing or water resistant resins and about 50% to about 80% by weight, preferably from about 65% to about 75% by weight, water. The corrugating adhesive may further comprise additives and fillers. The corrugating adhesives may be of the carrier and carrier no-carrier type.

Both natural unmodified starch and modified starch can be used. Any starch appropriate for use in corrugating adhesives may be used. Dextrins may also be used in the starch component of the invention. The corrugating adhesive may preferably comprise from about 18% to about 35% starch.

Unmodified starch is a commodity chemical produced from the root, stem or fruit from a number of plants. It is a high molecular weight carbohydrate polymer which is comprised of linear and branched polysaccharide polymers and it can have a moisture content from about 8% to about 20%, most commonly from about 11% to about 13%. Starches such as those derived from corn, wheat, barley, tapioca, potato and the like are suitable. Blends of starches from various sources also can be used. Pearl starches and powdered starches may be used.

The modified starch which is used in accordance with the invention can be mechanically, chemically or heat modified. Compared to unmodified starches, modified starches frequently possess superior physical properties such as increased solubility, better film forming, increase whiteness, improved gel strength, viscosity stability, increased adhesivity, improved resistance to shear and increased resistance to freeze-thaw degradation. Starches derived from other genetic forms of corn, such as high amylose and waxy corn, as well as sorghum varieties, would also be suitable. Chemically modified starches useful in the invention include modified oxidized starch such as hypochlorite-oxidized starch, acid thinned starches, cross-bonded starch and others which have reduced molecular weight, high fluidity and/or functional sub groups.

Examples of chemically modified starches which can be used in the invention and are commercially available are SUREBOND® Industrial Corn Starch or STABLEBOND® Industrial Corn Starch. These modified starches have residual carboxyl functionality and extreme uniformity and are available from Corn Products International, Inc., Westchester, Ill., USA (“Corn Products”). The corrugating adhesives may preferably comprise from about 1% to about 15% hydrocolloid and most preferably comprise from about 2% to about 8% hydrocolloid. In another preferred embodiment of the invention, the corrugating adhesives may comprise about 10% to about 35% hydrocolloid, i.e., hemicellulose, such as about 18% to about 30%.

The preferred hydrocolloid for use in the invention is hemicellulose. Hemicelluloses are described in U.S. Pat. No. 5,358,559 which is incorporated herein in its entirety by reference. The hemicellulose may be added to the corrugating adhesive or may be made in-situ by the addition of corn fiber when making the corrugating adhesive composition. The hemicellulose will preferably have a cellulose fraction, such as cellulose fractions obtained from corn fiber by alkali treatment that also contain bound hemicellulose.

Hydrocolloids with similar chemistry to hemicellulose may be used. These include hydrocolloids selected from the group consisting of gum arabic, xanthan gum, gum karaya, tragacanth, sodium alginates, carageenan, Guar gum, Locus bean gum, tara, pectins, gellan, cellulose derivatives such as carboxymethyl, methyl or ethyl cellulose, microcrystalline cellulose, or other polysaccharide type hydrocolloids can be used. Combinations of hydrocolloids may be used. The hydrocolloid may be chemically or enzymatically modified hydrocolloids, such as cellulose derivatives and enzymatically treated hemicellulose, like those referred to in U.S. Pat. No. 5,358,559 that are available under the trade name CELLACE from Nihon Shokuhin Kako Co. Ltd., Tokyo, JAPAN.

Starch hemicellulose blends may be used in place of or with the starch component to provide the corrugating adhesive composition with hemicellulose. These blends are particularly useful for use in the carrier portion of the invention. Starch hemicellulose blends available under the trademark FIBERBOND® Industrial Corn Starch, available from Corn Products, may be used in the invention.

The hemicellulose may be extracted from corn fiber in-situ when making the corrugating adhesive. Suitable corn fibers include crude fiber, typically described as feed, and more finished products such as dietary corn fiber which is made for human consumption. Crude fiber or feed generally contains from about 20% to about 40% hemicellulose and dietary corn fiber generally contains from about 50% to about 80% hemicellulose. When dietary corn fiber is employed, the amount used is preferably from 0.1 to about 5.0 parts per 100 parts of adhesive. The fiber in the adhesive, or in a carrier phase, preferably has a particle size of less than about 0.0005 inch and a geometric mean size of less than about 0.00005 inch.

Any strong base can be used for the caustic in the corrugating adhesives. Preferably, however, alkali metal hydroxides, such as sodium hydroxide or potassium hydroxide, are used. Combinations of caustics may also be used. The corrugating adhesives typically require an amount of caustic that depends on the amount of hydrocolloid added to achieve an equivalent gel temperature in the adhesive. Because the gel temperature will vary based on the amount and type of hydrocolloid used, the amount of caustic that should be used can be determined by measuring the final gel temperature of the adhesive. Another way to determine the amount of adequate caustic is to look for irreversible swelling of the adhesive in storage. Irreversible swelling is measured by viscosity rise which cannot be reversed by mixing or reheating adhesive that has dropped in temperature. The corrugating adhesive compositions may preferably comprise greater than 1% by weight caustic. Unless otherwise specified, the amount of caustic in the composition as set forth in this specification is by total weight of the components in the corrugating adhesive composition, i.e. by weight of the composition (by weight). Corrugating adhesives comprising caustic in amounts such as greater than 1% by weight to about 2.25% by weight, like about 1.25% by weight to about 2% by weight caustic are also within the scope of the invention. In embodiments of the invention, the composition comprises about 0.75% to about 2.25% by weight caustic and about 0.1% to about 4% by weight hemicellulose and a further embodiment concerns a corrugating adhesive composition comprising greater than 1% by weight caustic, including the ranges discussed above having greater than 1% by weight caustic, and about 4% to about 20% by weight hemicellulose.

The inventor has discovered that the critical constant for the concentration of caustic, based on the ratio of starch to caustic in the formulation to avoid undesired starch swelling in a corrugating adhesive composition can be increased by the inclusion of hydrocolloid, such as hemicellulose, in the composition. The concentration of caustic allowed in a corrugating adhesive composition is defined by the equation:

$$Caustic Concentration = \frac{( Amount of Caustic - {C}_{Critical} \times Amount of Starch )}{Amount of Water}$$

In the equation C_{Critical} is the critical constant which defines the maximum amount of caustic that can be included in the corrugating adhesive without the caustic causing swelling of the starch in the adhesive. In adhesive compositions that do not comprise hydrocolloid, i.e. hemicellulose which generally contains about 50% fiber or more, the critical constant is 1.6. However, when hydrocolloids are included the critical constant increases to above 1.6, such as in the range of greater than 1.6 to about 2.5, like greater than 1.6 to about 2.0, like about 1.7 to about 2.5 or 1.7 to about 2.0. Thus, the increase in the critical constant realized by the inclusion of hydrocolloid dictates that more caustic can be added to the corrugating adhesive composition and maintain a caustic concentration that will not result in undesired swelling of the starch by the caustic.

Any boron containing compound having free hydroxyl groups attached to the boron atoms can be used in the corrugating adhesives. The preferred compounds are commercial boric acid (ortho boric acid, H₃BO₃ and its hydrated forms H₃BO₃.H₂O) and borax (sodium tetraborate decahydrate, Na₂B₄O₇.10H₂O and other hydrate and anhydrous forms). The corrugating adhesive may preferably comprise from about 0.3% to about 0.8% boron containing compound.

The corrugating adhesive also comprises waterproofing or water resistant resins. Any waterproofing resins appropriate for corrugated boards may be used. Preferred waterproofing or water resistant resins include those which upon heating in basic media generate cross-linking species which react and cross-link with any available hydroxyl group in the starch, polyvinyl alcohol, hemicellulose or cellulose molecules. The cross-linking action reduces the hydrophilic nature and water-solubility of the starch, hemicellulose, and other polyhydroxy molecules by effectively removing the availability of hydroxyl groups to water and by developing hydrophobic, aliphatic cross-linking moieties. The waterproofing or water resistant resins may be incorporated into the corrugating adhesive when the adhesive is formulated or may be dosed into the adhesive prior to use on a corrugator.

Condensation products from the reaction of ketone and aldehyde compounds are suitable. These resins are characterized as polyether polymers, but can contain a variety of other monomers such as urea, melamine, and the like. Waterproofing resins available from the Harper/Love Adhesive Corporation, Charlotte, N.C., USA (“Harper/Love”) under the trade names AQUASEAL PLUS™, AQUASEAL™ and HYDRATITE™ may be used in the invention.

The corrugating adhesives may, optionally, include biocides. Any composition appropriate for retarding microbial growth in corrugating adhesives may be used in the invention. Preferred biocides are those available from Harper/Love under the HARLO-CIDE trade name. Biocides available from The Dow Chemical Company, Midland, Mich., USA under the trade name DOWCIL®, particularly DOWCIL®75, may also be used.

The corrugating adhesives may also include other additives and fillers, such as performance enhancing additives, including liquid additives, polyvinyl alcohol, latexes and the like. The hydrocolloids enhance the performance of many of these additives, particularly polyvinyl alcohol and latex emulsions based on acrylics and copolymers of styrene.

Carrier type corrugating adhesives of the invention can be made by separately preparing a carrier phase and a suspended phase and then combining the phases. The carrier phase is made by admixing the starch (or dextrin), hydrocolloid, caustic and water, in any order, while heating, after which the carrier is cooled. In lieu of starch and hydrocolloid, a starch and hemicellulose mixture, such as FIBERBOND® Industrial Corn Starch can be used. The suspended phase is made by admixing starch, the boron containing compound, the waterproofing or water resistant resins, and water while heating. The optional ingredients may be added to the suspended phase. The carrier phase is gradually added to the suspended phase with continuous mixing. Due to the ability of the hydrocolloid to absorb and buffer caustic, higher levels of caustic can be employed in this formulation than in traditional corrugating adhesives.

In an embodiment of the invention, hemicellulose from corn fiber is employed in a carrier type adhesive. The preferred corn fiber is dietary corn fiber, such as that available from dry millers containing from about 50% to about 80% hemicellulose and it can be prepared according to U.S. Pat. Nos. 4,994,115 or 5,073,201, which are incorporated herein in their entirety by reference. The corn fiber containing hemicellulose is admixed with the starch and water in the preparation of the carrier phase. The components are continuously mixed and heated to a temperature from about 100° F. (46° C.) to about 180° F. (82° C.). Sufficient caustic is then added to provide an alkaline pH on a gradual basis while continuing mixing and maintaining heating. If the hemicellulose is added in the form of corn fiber, mixing and heating are continued for a sufficient time to extract hemicellulose from the corn fiber. The pH should exceed about 10 and preferably will exceed about 12. The sufficient time and temperature will generally be from about 10 to about 40 minutes at from about 1000° F. (46° C.) to about 180° F. (82° C.). Lower temperatures generally correspond with longer times.

In a particular embodiment of the invention, the carrier type corrugating adhesive can be made by the following process:
1. Water, unmodified starch, and/or modified starch and/or dextrin, and corn fiber are mixed in a primary mixer and heated for at least about 1 minute and preferably from about 1 minute to about 25 minutes at a temperature of from about 100° F. (46° C.) to about 180° F. (82° C.);
2. An aqueous solution of caustic is added in an amount of about 0.5% to about to about 5% by weight of the starch on a dry basis to attain a pH from about 10 to about 14, preferably from about 12 to about 14, and mixing is continued for at least about 10 minutes and preferably from about 10 to about 40 minutes;
3. Additional water is added and mixing is continued for at least about 1 minute and preferably from about 1 to about 15 minutes, most preferably from about 5 to about 8 minutes, to make a carrier phase;
4. A secondary mixer is charged with water, heated to from about 70° F. (21° C.) to about 105° F. (41° C.), preferably from about 90° F. (32° C.) to about 100° F. (38° C.) and borax is added;
5. Unmodified starch and/or modified starch and/or dextrin and the waterproofing or water resistant resins are added, to make a suspended phase (also called a suspended starch phase), and the contents are mixed for from about 3 to about 25 minutes;
6. The contents of the primary mixer are gradually added to the secondary mixer with continuous mixing. This step typically is carried out over a period of about 5 to 20 minutes.

The invention also pertains to a method for bonding waterproof or chemically resistant papers, such as bonding coated corrugated media and/or liners to make waterproof and chemically resistant corrugated board comprising the step of joining a corrugated medium to at least one liner coated with a waterproofing or chemically resistant material using the corrugating adhesive described herein. The method may apply to all types of coated material. The adhesive is suited for bonding latex coated corrugated media and liner. Examples of such media and liners are those having a coating comprising one or more films of acrylic based resin compositions comprising zinc oxide to cross link the acrylic resins. Latex coated corrugating media and liner boards are described in U.S. Pat. Nos. 5,393,566; 5,429,294 and 5,858,173, the disclosures of which are incorporated herein in their entirety by reference.

A method for making a single-face corrugated board from one or more coated or uncoated corrugated media and one or more coated paper liners, such as latex coated material like those having one or more films of acrylic based resin compositions comprising zinc oxide to cross link the acrylic resins, comprises the steps of applying the adhesive composition of the invention onto the tips of the flutes to a first side of a sheet of corrugated medium having a first and second side each with a plurality of flutes comprising tips and then applying the coated paper liner by aligning the coated paper with the first side of the corrugated medium while simultaneously passing the coated paper liner and corrugating media through a corrugating roll and a pressure roll. A double-face portion can be made from one or more coated or uncoated corrugated media and one or more, preferably two or more, coated paper liners by applying the adhesive composition of the invention onto the tips of the flutes on the first side and on the second side of a sheet of the corrugated medium and then applying a first coated paper liner to the first side of the corrugated medium and a second coated paper liner to the second side of the corrugated medium by aligning the first sheet of coated paper with the first side of the corrugated medium and the second sheet of coated paper with the second side of the corrugated medium while simultaneously passing the coated paper and corrugated media through a corrugating roll and a pressure roll. The method for making the double-face portion may involve latex coated materials, like those having one or more films of acrylic based resin compositions comprising zinc oxide to cross link the acrylic resins. The adhesive can also be used on laminating and other gluing operations which involve heat and pressure curing of adhesives to bond papers together.

The corrugating adhesives of the invention exhibit improved bondability of coated papers, in particular complex latex coated media and linerboards. The hydrocolloid portion of the corrugating adhesive provides improved tack and bonding in the carrier portion of the adhesive. Also, in embodiments wherein the hydrocolloid is hemicellulose, the hemicellulose and the cellulose portion buffer and absorb caustic in the adhesive. This allows the adhesive to have higher caustic in the formula without prematurely gelling the secondary starch portion. The higher caustic improves bonding by reacting with the coating surface on the coated corrugated media and liner papers at the bond point, enabling penetration of the adhesive and waterproofing and water resistant resins into the paper surface.

The hydrocolloid will associate with the caustic thereby preventing the caustic from swelling the starch in the corrugating adhesive leaving it available to attack and etch the paper coatings which fosters bond strength which has been a problem in bonding coated papers with conventional adhesives. By incorporating hydrocolloid in the corrugating adhesives, particularly at the compositional amounts set forth herein, higher amounts of caustic can be used in the composition, alkali levels conventionally unattainable in corrugating adhesives. Thus, the corrugating adhesives of the invention are uniquely adapted to successfully bond coated papers because corrugating adhesive allows the adhesive to bond to paper fibers to themselves rather than having coating to coating bonding. Also, in embodiments of the invention involving waterproofing resins, these resins make the bond itself water resistant thus enhancing the performance of a corrugated container formed from coated board. If the adhesive is unable to bond to paper fibers, the resin will not make the bond waterproof and it will fail. Additionally, hydrocolloids, particularly hemicellulose, further improves bonding because of the linear nature of the polymer molecules of the hydrocolloid which improves the amount of penetration of the corrugating adhesive in small surface openings in the coatings on the coated paper.

### EXAMPLES

### Example 1

The carrier phase of a corrugating adhesive was made by adding 100 gallons of water into a primary mixer and heating the water to 150° F., adding 300 pounds of FIBERBOND® Industrial Corn Starch from Corn Products, and then adding 70 pounds of 50% solids liquid caustic and mixing for 25 minutes. One hundred gallons of cooling water were then added and the contents were mixed for 5 minutes. The suspended phase was made by adding 310 gallons of water into a secondary mixer and heating the water to 85° F. to 90° F. Nineteen pounds of 5 mol Borax and 1,200 pounds of pearl starch (Code 030050 from Corn Products) were then added to the secondary mixer and the contents were mixed for 5 minutes.

The corrugating adhesive was made by first gradually adding the carrier phase from the primary mixer into the secondary mixer over a period of 20 to 30 minutes and then mixing the combined carrier and suspended phases for 8 to 10 minutes. At the conclusion of mixing, the physical characteristics were measured and the corrugating adhesive was stored for trial runs. The physical characteristics of the corrugating adhesive are set forth in Table 1.

TABLE 1

| Viscosity | 55 secs. |
|---|---|
| Temperature | 100° F. |
| Gel Temp | 150° F. |
| Solids | |
| (d.b.) | 22.2% |
| (c.b.) | 24.8% |

Waterproofing resin was dosed into the adhesives prior to use on the corrugator. AQUASEAL™W50 from Harper/Love was added at a rate of 100 pounds per batch.

The corrugating adhesive composition was used to make corrugated board. M-Guard® moisture barrier linerboard (56#) from Liberty Paper Incorporated, Becker, Minn., USA was applied to both sides of 736B SPECTRA-GUARD® coated medium (26#) from Spectra-Kote, Gettysburg, Pa., USA (“Spectra-Kote”) in a corrugator operating at 308 feet per minute.

Samples of the board made with the adhesive of Example 1 were subjected to a dry adhesion test in accordance with the Technical Association of Pulp and Paper Industry, Inc. (“TAPPI”) 821 standard, and a wet adhesion test was run using the TAPPI 821 standard after soaking test samples for 24 hours in water. Also, samples were subjected to an edge crush test in accordance within the TAPPI 811 standard and a flat crush test in accordance with the TAPPI 825 standard. All of the standards are available from TAPPI, One Dunwoody Park, Atlanta, Ga., USA and are incorporated herein by reference. The results of these tests are set forth in Table 7 under Adhesive 1A and 2A.

### Example 2

In this Example separate corrugating adhesives were made for the single-face side of the corrugated board and for the double-face side of the board. The corrugating adhesives were the carrier type.

Single-Face Side Corrugating Adhesive

The carrier phase of the single-face side corrugating adhesive was made by adding 100 gallons of water into a primary mixer and heating the water to 140° F. Then, 144 pounds of FIBERBOND® Industrial Corn Starch from Corn Products, 95 pounds of pearl starch (Code 030050 from Corn Products) and 30 pounds of 50% solids liquid caustic were added to the primary mixer and the contents were mixed for 25 minutes. Ninety-five gallons of cooling water were then added and the contents were mixed for 5 minutes.

The suspended phase was made by adding 290 gallons of water into a secondary mixer and heating the water to 90° F. Ten pounds of 5 mol Borax, 1,200 pounds of pearl starch (Code 030050 from Corn Products), 1 pound of biocide and 6 ounces of the PLURONICO® L-61 anti-foaming agent from BASF, Mount Olive, N.J., USA, were then added to the secondary mixer and the contents were mixed for 5 minutes.

The single-face side corrugating adhesive was made by first gradually adding the carrier phase from the primary mixer into the secondary mixer over a period of 20 to 30 minutes and then mixing the combined carrier and suspended phases for 8 to 10 minutes. At the conclusion of mixing, the physical characteristics were measured and the corrugating adhesive was stored for trial runs. The physical characteristics of the single-face side corrugating adhesive are set forth in Table 2.

TABLE 2

| Viscosity | 55 secs. |
|---|---|
| Temperature | 100° F. |
| Gel Temp | 150° F. |
| Solids | |
| (d.b.) | 22.8% |
| (c.b.) | 25.3% |

Double-Face Side Corrugating Adhesive

The carrier phase of the double-face side corrugating adhesive was made by adding 100 gallons of water into a primary mixer and heating the water to 140° F. Then, 300 hundred pounds of FIBERBONDO® Industrial Corn Starch from Corn Products, 180 pounds of pearl starch (Code 030050 from Corn Products) and 42 pounds of 50% solids liquid caustic were added and the contents were mixed for 25 minutes. After 25 minutes the heat source was discontinued and 90 gallons of cooling water were added to the primary mixer and the contents were then mixed for 5 minutes.

The suspended phase was made by adding 290 gallons of water into a secondary mixer and heating the water to 90° F., then adding 10 pounds of 5 mol Borax, 1,300 pounds of pearl starch (Code 030050 from Corn Products) and 1 pound of biocide. The contents were mixed for 5 minutes to form the suspended phase.

The double-face side corrugating adhesive was made by first gradually adding the carrier phase from the primary mixer into the secondary mixer over a period of 20 to 30 minutes and then mixing the combined carrier and suspended phases for 8 to 10 minutes. At the conclusion of mixing, the physical characteristics were measured and the double-face side corrugating adhesive was stored for trial runs. The physical characteristics of the double-face side corrugating adhesive are set forth in Table 3.

TABLE 3

| Viscosity | 55 secs. |
|---|---|
| Temperature | 100° F. |
| Gel Temp | 145° F. |
| Solids | |
| (d.b.) | 25.4% |
| (c.b.) | 28.4% |

Waterproofing resin, AQUASEAL™ W150 from Harper/Love, was dosed into the finished adhesives at 100 pounds per completed batch prior to using on the corrugator.

The single-face side and double-face side corrugating adhesive compositions were used to make corrugated board. 736A SPECTRA-GUARD® coated liner (39#) from Spectra-Kote was applied to both sides of 736B SPECTRA-GUARD® coated medium (26#) from Spectra-Kote in a corrugator.

Samples of the board made with the adhesive of Example 2 were subjected to a dry adhesion test in accordance with the TAPPI 821 standard and wet adhesion was run using the TAPPI 821 standard after soaking test samples for 24 hours in water. Samples were also subjected to an edge crush test in accordance with the TAPPI 811 standard. The results of these tests are set forth in Table 7 under Adhesive 2B12, 2B14 and 2B18.

### Example3

A corrugating adhesive was made in a High Shear Mixer using 300 pounds water heated to 97° F., adding 54 pounds of FIBERBOND® Industrial Corn Starch from Corn Products and 14.8 pounds of 50% solids liquid caustic, mixing 5 minutes, adding 3.7 pounds 5 mol borax, mixing 3½ minutes, adding 430 pounds water and 235 pounds pearl starch (Code 030050 from Corn Products), and mixing 3½ minutes. The corrugating adhesive of this example had a viscosity of 29 secs., temperature of 106° F. and a gel temperature of 146° F.

Waterproofing resin, AQUASEAL™W150 from Harper/Love, was dosed into the finished adhesives at a rate of 20.5 pounds per 120 gallons of adhesive. Also, Liquid Additive XW200 from Harper/Love was added at a rate of 15 pounds per 120 gallons adhesive.

The corrugating adhesive composition was used to make corrugated board at speeds up to 500 feet per minute. The adhesive was applied to the corrugator to liner (69#) coated with Spectra-Guard 763A and medium (26#) coated with Spectra-Guard 763B. Samples of the board made with the adhesive of Example 3 were subjected to a dry adhesion test in accordance with the TAPPI 821 standard and wet adhesion was run using the TAPPI 821 standard after soaking test samples for 24 hours in water. Samples were also subjected to an edge crush test in accordance with the TAPPI 811 standard. The results are set forth in Table 4, sample 3A pertaining to board made at a speed of about 400 feet per minute and sample 3B pertaining to board made at a speed of about 500 feet per minute.

TABLE 4

| | Pin Adhesion - Lbs/LF | | | | Edge Crush | Flat Crush |
|---|---|---|---|---|---|---|
| | Dry T820 | | Wet T821 | | T811 | T825 |
| Adhesive | SF | DB | SF | DB | Lbs/LF | Lbs/SI |
| 1A | 72.4 | 63.4 | 5.5 | 5.60 | 59.3 | 41.1 |
| 2A | 74.5 | 64.4 | 7.6 | 8 | 61 | 40.6 |
| 2B12 | 83.2 | 72.1 | 4.9 | 7.5 | 44.3 | — |
| 2B14 | 79.3 | 72.3 | 7.4 | 7.9 | 43.9 | — |
| 2B18 | 90 | 77.8 | 8.6 | 7.3 | 48.1 | — |
| 3A | 84.8 | 76.1 | 2.6 | 5.2 | 59.1 | — |
| 3B | 77.8 | 66.9 | 2.0 | 5.8 | 68 | — |

### Example 4

This example demonstrates the effect of hydrocolloid (such as corn fiber or hemicellulose) on starch and the effect of the critical caustic level.

Twenty five grams of unmodified corn starch (030050, Corn Products) was treated with 0.77 grams sodium hydroxide in 75 grams water. This sample was stirred for 5 minutes and then allowed to settle for 24 hours in a 100 ml graduated cylinder. After 24 hours no starch settled, and the sample was a consistent fluid gel, demonstrating that the starch was swollen with the sodium hydroxide.

A sample in which 2 grams purified corn fiber from Bunge Milling, St. Louis, Mo., USA and 23 grams unmodified corn starch (030050, Corn Products) were combined and treated with 0.77 grams sodium hydroxide in 75 grams water was prepared. As in the experiment discussed above, the corn fiber and starch mixture was stirred for 5 minutes and then allowed to settle for 24 hours in a 100 ml graduated cylinder. The starch from the corn fiber and starch mixture settled to approximately 40 mis in the graduate. There was a low viscosity phase of corn fiber and a cloudy phase of extracted hemicellulose thus indicating that the caustic had not swelled the starch.

Further experimentation using the methods described above demonstrated that 25 grams of starch in 75 grams of water can be mixed with up to 0.63 grams of sodium hydroxide without swelling. By contrast, however, as shown above a 25 gram sample of starch and hemicellulose containing fiber can be mixed with more caustic without swelling the starch portion. In this example, 23 grams of starch and 2 grams of fiber are used as the starch portion. This is representative of a typical Stein Hall corrugating adhesive, where a portion of the total starch solids contains 8% corn fiber and able to produce 5% hemicellulose in situ. As demonstrated above, this blend can be treated with up to 0.77 grams sodium hydroxide without swelling the starch portion.

### Example 5

Slurries containing 25 pounds of corn fiber and starch mixtures having varying ratios of fiber to starch in 75 pounds of water were prepared and analyzed as discussed in Example 4 to determine the amount of caustic that can be included at varying concentrations of fiber without swelling the starch. The data is presented in Table 5 showing the amount of caustic added prior to starch swelling.

TABLE 5

| | Amount of Caustic Added |
|---|---|
| Amount of Fiber | Prior to Starch Swelling |
| 0.00% | 0.63 |
| 5.88% | 0.75 |
| 11.76% | 0.81 |
| 17.65% | 0.88 |

Hemicellulose contains about 50% fiber and, thus, to obtain the fiber contents set forth in Table 5, the compositions must comprise hemicellulose in amounts about two times the amount of fiber set forth in Table 5, i.e., hemicellulose in amounts of about 11.76%, about 23.52% and about 35.30%.

What is claimed is:

1. A corrugating adhesive for bonding coated media or liner comprising starch, hydrocolloid, caustic, waterproofing resin or water resistant resin and water wherein the concentration of caustic in the corrugating adhesive is defined by the equation (Amount of Caustic−C_{Critical}×Amount of Starch)/Amount of Water and C_{Critical} is greater than 1.6.

2. The corrugating adhesive of claim 1 wherein the C_{Critical} is greater than 1.6 to about 2.5.

3. The corrugating adhesive of claim 1 comprising from about 15% to about 45% by weight starch, about 1% to about 40% by weight hydrocolloid, about 0.75% to about 2.25% by weight caustic, up to about 2% by weight boron containing compound, about 0.5% to about 5% by weight waterproofing resin or water resistant resin and about 50% to about 80% by weight water.

4. The corrugating adhesive of claim 3 wherein the hydrocolloid is hemicellulose and the amount of hemicellulose is from about 0.1% to about 4% by weight.

5. The corrugating adhesive of claim 3 wherein the amount of hydrocolloid is about 2% to about 8% by weight.

6. The corrugating adhesive of claim 3 wherein the amount of boron containing compound is about 0.3% to about 0.8% by weight.

7. The corrugating adhesive of claim 1 wherein the amount of caustic is greater than 1% by weight.

8. The corrugating adhesive of claim 7 wherein the amount of caustic is from about 1.25% to about 2.00% by weight.

9. The corrugating adhesive of claim 7 wherein the hydrocolloid is hemicellulose and the amount of hemicellulose is from about 4% to about 20% by weight.

10. The corrugating adhesive of claim 1 having a pH of between about 10 and about 14.

11. The corrugating adhesive of claim 1 of the carrier or carrier no-carrier type.

12. The corrugating adhesive of claim 1 wherein the starch is selected from the group consisting of corn, wheat, barley, tapioca, potato and combinations thereof.

13. The corrugating adhesive of claim 1 wherein the starch is modified starch.

14. The corrugating adhesive of claim 13 wherein the modified starch is selected from the group consisting of high amylose corn starch, waxy corn starch, acid thinned starch, etherified starch, esterified starch, hypochlorite-oxidized starch and cross-bonded starch.

15. The corrugating adhesive of claim 1 wherein the hydrocolloid is selected from the group consisting of hemicellulose, gum arabic, xanthan gum, gum karaya, tragacanth, sodium alginates, carageenan, Guar gum, Locus bean gum, tara, pectins, gellan, cellulose derivative, microcrystalline cellulose or combinations thereof.

16. The corrugating adhesive of claim 15 wherein the hemicellulose is from in-situ extraction of corn fiber.

17. The corrugating adhesive of claim 15 wherein the cellulose derivative is selected from the group of carboxymethyl cellulose, methyl cellulose and ethyl cellulose.

18. The corrugating adhesive of claim 1 wherein the caustic comprises alkali metal hydroxides.

19. The corrugating adhesive of claim 1 wherein the waterproofing resin is a condensation product from the reaction of a ketone and an aldehyde compound.

20. A method of making waterproof or chemically resistant corrugated board comprising joining by bonding a coated corrugated board to at least one water proof or chemically resistant liner using the corrugating adhesive of claim 1.
